SEQUENCE LISTING (6 sequences)

SEQ ID NO: 1 - DNA, 1000 bp, Arabidopsis thaliana
  misc_feature (1)..(1000): Ceres Promoter PT0623
  misc_feature (1)..(1000): Promoter Candidate ID 11768718
aaagttattgacattttgaaaggaccgtaaatattaccaaaaaactgacggagttaggatcggccacgtagaaagggacaaagagagaacagtcacggactcggccagactaagtatgggcctgtctgaatccaaactcagctaagttccaaaagcataaagagagatgtgtaatgaaatgaacgtattctagaaacgaaagcaatgttatgctttgtttttgagccacatgtttttgggagatggagagaatcttttttacgtttttaacctaacccacttggcacttggccaaaaaagtgagaagaaactgtggcgaatgagtaggccacgccatggactttgttccttgtccttcaaaagttaaatttatgttatgcgtggggacaatctaagcaacgtggttcctttaaatatcgcagcttcctcttttacacttttggagcctacgtgttttgttttggaccggccaaatacacgagtcagtcagtttagaaataatttggatgtccaaaaatcttggagatccaaataaaataattagcatgttttagttcataagaatatgaaatgtagataaactgtctatattaatttttccatagaattggctttttatcgaggtgatgtacttaatgactttgttgattactactcgtataacaataaagaatatgatactatgtgagacttataatgaatttggtgtgtgttaattaatccagttgaaacagtttaataacaaatcagaataaaaattgtagtaagaaaatttgaacgctgatccttcaacctagatagtgaacctttcaaatactatatgattcacgtgtaatgtttttgaccgttggttatttttgtgtgaactatattaacttatcaatatcgaaaggctaaataagtaaataactaaaagaaagttcaggaaacaactcgacctaatgacctatcatttctgatcacccgtcctataaatacatacgtaagatcattcgttact

SEQ ID NO: 2 - DNA, 1000 bp, Arabidopsis thaliana
  misc_feature (1)..(1000): Ceres Promoter YP0396
  misc_feature (1)..(1000): Promoter Candidate ID 11768788
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 3 - DNA, 1000 bp, Arabidopsis thaliana
  misc_feature (1)..(1000): Ceres Promoter YP0377
  misc_feature (1)..(1000): Promoter Candidate ID 11768593
tataaaccattcctataacaccatatttaaacataacaatgaattgcttggatttcaaactttattaaatttggattttaaattttaatttgattgaattatacccccttaattggataaattcaaatatgtcaactttttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaactgctggcaaccacttttgccgcgtttaatttctttctgaggcttatataaatagatcaaaggggaaagtgagatataatacagacaaaacaagagaaaaga

SEQ ID NO: 4 - DNA, 14 bp, Arabidopsis thaliana
  misc_feature (1)..(14): transcription activator recognition site Zinc finger protein MIG1
ggtaaaaatgcggg

SEQ ID NO: 5 - DNA, 16 bp, Arabidopsis thaliana
  misc_feature (1)..(16): transcription activator recognition site ABF1
tnncgtnnnnnntgat

SEQ ID NO: 6 - DNA, 11 bp, Arabidopsis thaliana
  misc_feature (1)..(11): transcription activator recognition site UME6
tsggcggctaw

(19) United States
(12) Patent Application Publication
(10) Pub. No.: US 2008/0104726 A1
(43) Pub. Date: May 1, 2008

(54) FRUIT REGULATORY REGIONS

(75) Inventors: Joon-Hyun Park, Oak Park, CA (US); Steven Craig Bobzin, Malibu, CA (US); Courtney A. Becker, Phoenix, AZ (US); Yiwen Fang, Los Angeles, CA (US); Zhihong C. Cook, Aliso Viejo, CA (US); Michael F. Portereiko, Thousand Oaks, CA (US)

Correspondence Address: FISH & RICHARDSON P.C., P.O. BOX 1022, MINNEAPOLIS, MN, 55440-1022, US

(73) Assignee: CERES, INC., Thousand Oaks, CA (US)

(21) Appl. No.: 11/867,537

(22) Filed: Oct. 4, 2007

Related U.S. Application Data
(60) Provisional application No. 60/849,502, filed on Oct. 5, 2006.

(51) Int. Cl. C12N 15/82; C12N 15/11; C12N 15/00; C12Q 1/68; A01H 5/00

(52) U.S. Cl. 800/278; 536/23.6; 435/320.1; 800/298; 435/6

(57) ABSTRACT

Regulatory regions suitable for directing expression of a heterologous polynucleotide in fruit tissues, e.g., flesh and peel tissues, of plants are described, as well as nucleic acid constructs that include these regulatory regions. Also disclosed are transgenic plants that contain such constructs and methods of producing such transgenic plants.

### CROSS-REFERENCE TO RELATED APPLICATIONS

This application claims priority under 35 U.S.C. 119(e) to U.S. Application Ser. No. 60/849,502, filed on Oct. 5, 2006, the entire contents of which are hereby incorporated by reference.

### BACKGROUND

1. Technical Field

This document relates to methods and materials involved in regulating gene expression in eukaryotic organisms (e.g., plants).

2. Background Information

An essential element for genetic engineering of plants is the ability to express genes using various regulatory regions. The expression pattern of a transgene, conferred by a regulatory region, is critical for the timing, location, and conditions under which a transgene is expressed, as well as the intensity with which the transgene is expressed in a transgenic plant. Having the ability to modulate the pattern and level of expression of a transgene can allow plants with desired characteristics or traits to be generated. There is a continuing need for suitable regulatory regions that can facilitate transcription of sequences that are operably linked to the regulatory region.

### SUMMARY

This document provides materials and methods involving regulatory regions having the ability to direct transcription in eukaryotic organisms (e.g., plants). For example, this document provides regulatory regions having the ability to direct transcription in fruit tissues of plants, such as tomato plants. Also provided herein are nucleic acid constructs, plant cells, and plants containing such regulatory regions, and methods of using such regulatory regions to express polynucleotides in plants and to alter the phenotype of plant cells. Regulatory regions that direct transcription in fruit can be used, for example, to modulate (e.g., increase or decrease) the nutritional or caloric value of fruit, or to modulate the response of fruit to stress, pathogens, herbicides, bruising, or mechanical agitation. In some embodiments, regulatory regions that direct transcription in fruit can be used to express polypeptides involved in flavonoid, stilbene, coumarin, phytosterol, terpenoid, and/or monoterpenoid biosynthesis.

In one aspect, an isolated nucleic acid is provided that has 90% or greater sequence identity (e.g., 95% or greater sequence identity or 99% or greater sequence identity) to a polynucleotide sequence selected from the group consisting of a) nucleotides 500 to 1000 of SEQ ID NO:2, b) nucleotides 350 to 1000 of SEQ ID NO:2, and c) nucleotides 234 to 1000 of SEQ ID NO:2. The nucleic acid is 500 to 800 nucleotides in length and has the ability to direct transcription in fruit tissues of a plant.

A nucleic acid construct also is featured that includes a regulatory region operably linked to a heterologous polynucleotide. The regulatory region is 500 to 800 nucleotides in length and has 90% or greater sequence identity (e.g., 95% or greater sequence identity, or 99% or greater sequence identity) to the polynucleotide sequence selected from the group consisting of a) nucleotides 500 to 1000 of SEQ ID NO:2, b) nucleotides 350 to 1000 of SEQ ID NO:2, and c) nucleotides 234 to 1000 of SEQ ID NO:2. The heterologous polynucleotide can include a nucleotide sequence encoding a polypeptide (e.g., a resveratrol synthase polypeptide or an enzyme involved in flavonoid biosynthesis, phytosterol biosynthesis, or monoterpenoid biosynthesis). The heterologous polynucleotide can be in an antisense orientation relative to the regulatory region. The heterologous polynucleotide can be transcribed into an interfering RNA.

In another aspect, a transgenic plant or plant cell is featured that includes a nucleic acid construct. The nucleic acid construct includes a regulatory region operably linked to a heterologous polynucleotide, wherein the regulatory region is 500 to 800 nucleotides in length and has 90% or greater sequence identity to the polynucleotide sequence selected from the group consisting of a) nucleotides 500 to 1000 of SEQ ID NO:2, b) nucleotides 350 to 1000 of SEQ ID NO:2, and c) nucleotides 234 to 1000 of SEQ ID NO:2. The plant or plant cell can be from a genus selected from Capsicum, Fragaria, Lycopersicon, Solanum, Vaccinium, or Vitis.

A transgenic plant or plant cell also is provided that includes (a) a first nucleic acid that includes a regulatory region operably linked to a heterologous polynucleotide encoding a transcription activator polypeptide, wherein the regulatory region is 500 to 800 nucleotides in length and has 90% or greater sequence identity to the polynucleotide sequence selected from the group consisting of i) nucleotides 500 to 1000 of SEQ ID NO:2, ii) nucleotides 350 to 1000 of SEQ ID NO:2, and iii) nucleotides 234 to 1000 of SEQ ID NO:2, and (b) a second nucleic acid that includes a sequence of interest operably linked to a recognition site for the transcription activator polypeptide. The sequence of interest can encode a polypeptide (e.g., a resveratrol synthase polypeptide or an enzyme involved in flavonoid biosynthesis, phytosterol biosynthesis, or monoterpenoid biosynthesis). The first and second nucleic acids can be present on the same nucleic acid construct. The plant or plant cell can be from a genus selected from Capsicum, Fragaria, Lycopersicon, Solanum, Vaccinium, or Vitis.

In another aspect, a nucleic acid construct is provided that includes a regulatory region having 80% or greater sequence identity to the polynucleotide sequence set forth in SEQ ID NO:1, SEQ ID NO:2, or SEQ ID NO:3 operably linked to a heterologous polynucleotide. The sequence identity can be 90%, 95%, or 99% or greater. The heterologous polynucleotide can comprise a nucleotide sequence encoding a polypeptide. The polypeptide can be a resveratrol synthase polypeptide. The polypeptide can be an enzyme involved in flavonoid biosynthesis. The polypeptide can be an enzyme involved in phytosterol biosynthesis. The polypeptide can be an enzyme involved in monoterpenoid biosynthesis. The heterologous polynucleotide can be in an antisense orientation relative to the regulatory region. The heterologous polynucleotide can be transcribed into an interfering RNA. A transgenic plant or plant cell that includes such a nucleic acid construct also is provided.

In yet another aspect, a transgenic plant or plant cell is provided. The transgenic plant or plant cell comprises (a) a first nucleic acid comprising a regulatory region having 80% or greater sequence identity (e.g., 90% or greater sequence identity) to the polynucleotide sequence set forth in SEQ ID NO:1, SEQ ID NO:2, or SEQ ID NO:3 operably linked to a heterologous polynucleotide encoding a transcription activator polypeptide, and (b) a second nucleic acid comprising a sequence of interest operably linked to a recognition site for the transcription activator polypeptide. The sequence of interest can encode a polypeptide. The polypeptide can be a resveratrol synthase polypeptide. The first and second nucleic acid can be present on the same nucleic acid construct. The plant or plant cell can be from a genus selected from Capsicum, Fragaria, Lycopersicon, Solanum, Vaccinium, or Vitis.

In another aspect, a method of producing a transgenic plant is provided. The method comprises (a) introducing into a plant cell a nucleic acid construct described above; and (b) growing a plant from the plant cell.

In another aspect, a method of identifying whether or not a regulatory region has a desired expression profile in an organism of interest is provided. The method comprises: (a) transforming a model organism with an isolated nucleic acid comprising a first regulatory region operably linked to a reporter gene, the first regulatory region having the desired expression profile in the organism of interest; (b) selecting a second regulatory region having an expression profile in the model organism that is similar to the expression profile of the first regulatory region in the model organism; and (c) determining the expression profile of the second regulatory region in the organism of interest, thereby identifying whether or not the second regulatory region has the desired expression profile in the organism of interest.

In another aspect, a transgenic plant is provided. The transgenic plant comprises a nucleic acid construct comprising a regulatory region having 80% or greater sequence identity (e.g., 90% or greater sequence identity) to the polynucleotide sequence set forth in SEQ ID NO:1, SEQ ID NO:2, or SEQ ID NO:3 operably linked to a heterologous polynucleotide, where the regulatory region preferentially directs transcription of the heterologous polynucleotide in fruit of the plant. The regulatory region can preferentially direct transcription in peel tissue of the fruit. The regulatory region can preferentially direct transcription in fleshy tissue of the fruit.

In another aspect, a method of making a transgenic plant is provided. The method comprises: a) transforming plant cells with a nucleic acid construct described above; and b) identifying a plant transformed with the nucleic acid construct, thereby producing the transgenic plant. Step b) can comprise identifying a plurality of plants transformed with the nucleic acid construct. The identifying step can include selecting a transgenic plant in which the regulatory region preferentially directs transcription of the heterologous polynucleotide in fruit of the plant.

A method of expressing a polynucleotide in fruit tissue also is provided. The method comprises growing a plant under conditions in which fruit are formed, where the plant comprises a nucleic acid construct comprising a regulatory region having 80% or greater sequence identity (e.g., 90% or greater sequence identity) to the polynucleotide sequence set forth in SEQ ID NO:1, SEQ ID NO:2, or SEQ ID NO:3 operably linked to a heterologous polynucleotide, and where the regulatory region preferentially directs transcription of the heterologous polynucleotide in fruit of the plant. The regulatory region can preferentially direct transcription of the heterologous polynucleotide in fleshy tissue of the fruit. The regulatory region can preferentially direct transcription of the heterologous polynucleotide in peel tissue of the fruit. The plant can be from the Solanaceae family. The plant can be a tomato plant. The regulatory region can preferentially direct transcription of the heterologous polynucleotide in peel tissue of the fruit. The regulatory region can preferentially direct transcription of the heterologous polynucleotide in fleshy tissue of the fruit. The transcription can occur during mature green stages, breaker stage, or ripening stages of the fruit.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

The invention features isolated nucleic acids comprising regulatory regions. The terms “nucleic acid” and “polynucleotide” are used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded, i.e., a sense strand or an antisense strand, Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An isolated nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences, e.g., a chemically synthesized nucleic acid, or a cDNA or genomic DNA fragment produced by the polymerase chain reaction (PCR) or restriction endonuclease treatment. An isolated nucleic acid also refers to a DNA molecule that is incorporated into a vector, an autonomously replicating plasmid, or a virus, or transformed into the genome of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Regulatory Regions

A regulatory region described herein is a nucleic acid that can direct transcription when the regulatory region is operably linked 5′ to a heterologous nucleic acid. As used herein, “heterologous nucleic acid” refers to a nucleic acid other than the naturally occurring coding sequence to which the regulatory region was operably linked in a plant. With regard to one regulatory region provided herein, PT0623 (SEQ ID NO:1), a heterologous nucleic acid is a nucleic acid other than the coding sequence for the chlorophyll A-B binding family protein/early light-induced protein (ELIP) from Arabidopsis. With regard to another regulatory region provided herein, YP0396 (SEQ ID NO:2), a heterologous nucleic acid is a nucleic acid other than the coding sequence for the putative photoassimilate-responsive protein from Arabidopsis. With regard to another regulatory region provided herein, YP0377 (SEQ ID NO:3), a heterologous nucleic acid is a nucleic acid other than the Arabidopsis genomic Iocus At1g07135 encoding a glycine-rich protein. The term “operably linked” refers to positioning of a regulatory region and a transcribable sequence in a nucleic acid so as to allow or facilitate transcription of the transcribable sequence. For example, a regulatory region is operably linked to a coding sequence when RNA polymerase is able to transcribe the coding sequence into mRNA, which then can be translated into a protein encoded by the coding sequence.

Regulatory regions can include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, and introns.

The nucleic acid sequences set forth in SEQ ID NOs:1-3 are examples of regulatory regions provided herein. However, a regulatory region can have a nucleotide sequence that deviates from that set forth in SEQ ID NO:1, SEQ ID NO:2, or SEQ ID NO:3, while retaining the ability to direct expression of an operably linked nucleic acid. For example, a regulatory region having 80% or greater (e.g., 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, or 99% or greater) sequence identity to the nucleotide sequence set forth in SEQ ID NO:1, SEQ ID NO:2, or SEQ ID NO: 3 can direct expression of an operably linked nucleic acid.

The term “percent sequence identity” refers to the degree of identity between any given query sequence, e.g., SEQ ID NO:1, and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A percent identity for any subject nucleic acid relative to a query nucleic acid can be determined as follows. A query nucleic acid sequence is aligned to one or more subject nucleic acid sequences using the computer program ClustalW (version 1.83, default parameters), which allows alignments of nucleic acid sequences to be carried out across their entire length (global alignment). Chenna et al., Nucleic Acids Res., 31(13):3497-500 (2003). ClustalW calculates the best match between a query and one or more subject sequences, and aligns them so that identities, similarities, and differences can be determined. Gaps of one or more residues can be inserted into a query sequence, a subject sequence, or both, to maximize sequence alignments. For fast pairwise alignment of nucleic acid sequences, the following parameters are used: word size: 2; window size: 4; scoring method: percentage; number of top diagonals: 4; and gap penalty: 5. For alignment of multiple nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; and gap penalty: 3. For multiple alignment of protein sequences, the following parameters are used: weight matrix: blosum; gap opening penalty: 10.0; gap extension penalty: 0.05; hydrophilic gaps: on; hydrophilic residues: G, P, S, N, D, Q, E, R, K; and residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site (ebi.ac.uk/clustalw).

To determine a percent identity between a query sequence and a subject sequence, ClustalW divides the number of identities in the best alignment by the number of residues compared (gap positions are excluded), and multiplies the result by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

Fragments of a regulatory region described herein can be made that retain the ability to preferentially direct transcription in fruit tissue. For example, fragments of the regulatory region of SEQ ID NO:2 that are about 500 to about 800 (e.g., 525, 550, 575, 600, 625, 650, 675, 700, 725, 750, 775, 790, 795) nucleotides in length can be made. In particular, fragments containing nucleotides 500 to 1000 of SEQ ID NO:2, nucleotides 350 to 1000 of SEQ ID NO:2, or nucleotides 234 to 1000 of SEQ ID NO:2 can preferentially direct transcription in fruit tissue. See Example 4. Fragments of the regulatory region of SEQ ID NO:1 and SEQ ID NO:3 also can be made. For example, with respect to the regulatory region of SEQ ID NO:1, a fragment at least 800 (850, 875, 900, 925, 950, 975, or 999) nucleotides in length can be made. For example, a fragment containing nucleotides 200 to 1000 of SEQ ID NO:1 may preferentially direct transcription in fruit tissue.

A regulatory region featured herein can be made by cloning 5′ flanking sequences of an ELIP gene, a gene encoding a putative photoassimilate-responsive protein, or a gene encoding a glycine-rich protein. Alternatively, a regulatory region can be made by chemical synthesis and/or PCR technology. PCR refers to a technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. PCR is described, for example, in PCR Primer: A Laboratory Manual, Ed. by Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplification. See, for example, Lewis, Genetic Engineering News, 12(9):1 (1992); Guatelli et al., Proc. Natl. Acad. Sci. USA, 87:1874-1878 (1990); and Weiss, Science, 254:1292 (1991). Various lengths of a regulatory region described herein can be made by similar techniques. A regulatory region also can be made by ligating together fragments of various regulatory regions. Methods for ligation of nucleic acid fragments, including PCR fragments, are known to those of ordinary skill in the art. PCR strategies also are available by which site-specific nucleotide sequence modifications can be introduced into a template nucleic acid.

The ability of a regulatory region to direct expression of an operably linked nucleic acid can be assayed using methods known to one having ordinary skill in the art. In particular, regulatory regions of varying lengths and regulatory regions comprising combinations of various regulatory regions ligated together can be operably linked to a reporter nucleic acid and used to transiently or stably transform a cell, e.g., a plant cell. Suitable reporter nucleic acids include β-glucuronidase (GUS), green fluorescent protein (GFP), yellow fluorescent protein (YFP), and luciferase (LUC). Expression of the gene product encoded by the reporter nucleic acid can be monitored in such transformed cells using standard techniques.

When a heterologous nucleic acid is operably linked to a cell-, tissue-, or organ-preferential regulatory region, transcription occurs only or predominantly in a particular cell type, tissue, or organ, respectively. For example, a regulatory region can drive expression preferentially in the fruit of a plant. A fruit is the ripened ovary, together with the seeds, of a flowering plant. In many species, the fruit incorporates the ripened ovary and surrounding tissues. Fruits are the means by which flowering plants disseminate seeds. Many foods are botanically fruits, but are treated as vegetables in cooking. These include cucurbits (e.g., squash and pumpkin), maize, tomato, cucumber, aubergine (eggplant), and sweet pepper, along with nuts, and some spices, such as allspice, nutmeg and chilies. A regulatory region described herein drives expression preferentially in fruit tissue of a plant. Fleshy tissue of tomato includes pericarp, placenta and columella tissue. In some embodiments, a regulatory region described herein directs transcription primarily in cell layers of the peel of the fruit of a plant. For example, the YP0396 regulatory region directs transcription primarily in cell layers of the peel of tomato fruit. A regulatory region described herein typically directs transcription during a number of developmental stages of fruit ripening. For example, a regulatory region described herein typically directs transcription during one or more of the mature green stages, breaker stage, or ripening stages of tomato fruit.

Nucleic Acid Constructs

Nucleic acid constructs containing nucleic acids such as those described herein also are provided. A nucleic acid construct can be a vector. A vector is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, Backs, Yaks, or PACs. The term “vector” includes cloning, transformation, and expression vectors, as well as viral vectors and integrating vectors. An expression vector is a vector that includes one or more regulatory regions. Suitable expression vectors include, without limitation, plasmids and viral vectors derived from, for example, bacteriophage, baculoviruses, and retroviruses. Numerous vectors and expression systems are commercially available from such corporations as Novae (Madison, Wis.), Clutch (Mountain View, Calif.), Strata gene (La Jolla, Calif.), and Invitrogen/Life Technologies (Carlsbad, Calif.).

A nucleic acid construct includes a regulatory region as disclosed herein. A construct also can include a heterologous nucleic acid operably linked to the regulatory region, in which case the construct can be introduced into an organism and used to direct expression of the operably linked nucleic acid. The heterologous nucleic acid can be operably linked to the regulatory region in the sense or antisense orientation. In some embodiments, a heterologous nucleic acid is linked to a regulatory region in the sense orientation and transcribed and translated into a polypeptide. The regulatory region can be operably linked from approximately 1 to 150 nucleotides upstream of the ATG translation start codon of a heterologous nucleic acid in the sense orientation. For example, the regulatory region can be operably linked 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 130 nucleotides, 140 nucleotides, or 150 nucleotides upstream of the ATG translation start codon of a heterologous nucleic acid in the sense orientation. In some cases, the regulatory region can be operably linked from approximately 151 to 500 nucleotides upstream of the ATG translation start codon of a heterologous nucleic acid in the sense orientation. In some cases, the regulatory region can be operably linked from approximately 501 to 1125 nucleotides upstream of the ATG translation start codon of a heterologous nucleic acid in the sense orientation.

A nucleic acid construct may contain more than one regulatory region. In some embodiments, each regulatory region is operably linked to a heterologous nucleic acid. For example, a nucleic acid construct may contain two regulatory regions, each operably linked to a different heterologous nucleic acid. The two regulatory regions can be the same or different, and one or both of the regulatory regions in such a construct can be a regulatory region described herein. A nucleic acid construct also can contain inducible elements, intron sequences, enhancer sequences, insulator sequences, or targeting sequences other than those present in a regulatory region described herein. Regulatory regions and other nucleic acids can be incorporated into a nucleic acid construct using methods known in the art.

Nucleic-Acid Based Methods for Inhibition of Gene Expression

A nucleic acid construct may include a heterologous nucleic acid that is transcribed into RNA useful for inhibiting expression of a gene. A number of nucleic acid based methods, including antisense RNA, ribozyme directed RNA cleavage, post-transcriptional gene silencing (PTGS), e.g., RNA interference (RNAi), and transcriptional gene silencing (TGS), can be used to inhibit gene expression in plants. Antisense technology is one well-known method. In this method, a nucleic acid segment from a gene to be repressed is cloned and operably linked to a regulatory region and a transcription termination sequence so that the antisense strand of RNA is transcribed. The recombinant vector is then transformed into plants, as described below, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence of a polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the polypeptide of interest, and can have a length that is shorter, the same as, or longer than the corresponding length of the sense sequence. In some cases, one strand of the stem portion of a double stranded RNA comprises a sequence that is similar or identical to the 3′ or 5′ untranslated region of the mRNA encoding the polypeptide of interest, and the other strand of the stem portion of the double stranded RNA comprises a sequence that is similar or identical to the sequence that is complementary to the 3′ or 5′ untranslated region, respectively, of the mRNA encoding the polypeptide of interest. In other embodiments, one strand of the stem portion of a double stranded RNA comprises a sequence that is similar or identical to the sequence of an intron in the pre-mRNA encoding the polypeptide of interest, and the other strand of the stem portion comprises a sequence that is similar or identical to the sequence that is complementary to the sequence of the intron in the pre-mRNA. The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron. A double stranded RNA can have zero, one, two, three, four, five, six, seven, eight, nine, ten, or more stem-loop structures. A construct including a sequence that is operably linked to a regulatory region and a transcription termination sequence, and that is transcribed into an RNA that can form a double stranded RNA, is transformed into plants as described below. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing regulatory regions operably linked to nucleic acid molecules in sense orientation can also be used to inhibit the expression of a gene. The transcription product can be similar or identical to the sense coding sequence of a polypeptide of interest. The transcription product can also be unpolyadenylated, lack a 5′ cap structure, or contain an unsplicable intron, Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA, or an intron in a pre-mRNA encoding a polypeptide of interest. In some embodiments, the sense or antisense sequence is identical or complementary to a sequence of the regulatory region that drives transcription of the gene encoding a polypeptide of interest. In each case, the sense sequence is the sequence that is complementary to the antisense sequence.

The sense and antisense sequences can be any length greater than about 12 nucleotides (e.g., 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence encoding an expansin polypeptide. The sense sequence complementary to the antisense sequence can be a sequence present within the mRNA of the expansin polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have any arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

Expression of a Sequence of Interest

A regulatory region described herein can be used to direct tissue (e.g., fruit) preferential expression of a sequence of interest operably linked to the regulatory region. A sequence of interest operably linked to a regulatory region can encode a polypeptide or can regulate the expression of a polypeptide. A sequence of interest that encodes a polypeptide can encode a plant polypeptide, a non-plant polypeptide, e.g., a mammalian polypeptide, a modified polypeptide, a synthetic polypeptide, or a portion of a polypeptide. In some embodiments, a sequence of interest is transcribed into an anti-sense or interfering RNA molecule.

In some embodiments, a sequence of interest can encode an enzyme involved in flavonoid biosynthesis, such as naringenin-chalcone synthase (EC 2.3.1.74), polyketide reductase, chalcone isomerase (EC 5.5.1.6), flavanone 4-reductase (EC 1.1.1.234), dihydrokaempferol 4-reductase (EC 1.1.1.219), flavone synthase (EC 1.14.11.22), flavone 7-O-beta-glucosyltransferase (EC 2.4.1.81), flavone apiosyltransferase (EC 2.4.2.25), isoflavone-7-O-beta-glucoside 6″-O-malonyltransferase (EC 2.3.1.115), apigenin 4′-O-methyltransferase (EC 2.1.1.75), flavonoid 3′-monooxygenase (EC 1.14.13.21), luteolin O-methyltransferase (EC 2.1.1.42), flavonoid 3′,5′-hydroxylase (EC 1.14.13.88), 4′-methoxyisoflavone 2′-hydroxylase (EC 1.14.13.53), isoflavone 4′-O-methyltransferase (EC 2.1.1.46), flavanone 3-dioxygenase (EC 1.14.11.9), leucocyanidin oxygenase (EC 1.14.11.19), flavonol synthase (EC 1.14.11.23), 2′-hydroxyisoflavone reductase (EC 1.3.1.45), leucoanthocyanidin reductase (EC 1.17.1.3), anthocyanidin reductase (EC 1.3.1.77), flavonol 3-O-glucosyltransferase (EC 2.4.1.91), quercetin 3-O-methyltransferase (EC 2.1.1.76), anthocyanidin 3-O-glucosyltransferase (EC 2.4.1.115), flavonol-3-O-glucoside L-rhamnosyltransferase (EC 2.4.1.159), UDP-glucose:anthocyanin 5-O-glucosyltransferase (2.4.1.-), or anthocyanin acyltransferase (2.3.1.-).

In some embodiments, a sequence of interest can encode an enzyme involved in stilbene synthesis such as trihydroxystilbene synthase (EC 2.3.1.95) or an oxidoreductase (EC 1.14.-.-).

In some embodiments, a sequence of interest can encode an enzyme involved in coumarin synthesis such as trans-cinnamate 2-monooxygenase (EC 1.14.13.14), 2-coumarate O-beta-glucosyltransferase (EC 2.4.1.114), a cis-trans-isomerase (EC 5.2.1.-), or a beta-glucosidase (EC 3.2.1.21).

In certain cases, a sequence of interest encodes an enzyme involved in phytosterol biosynthesis such as 4-hydroxy-3-methylbut-2-enyl diphosphate reductase (EC 1.17.1.2), 4-hydroxy-3-methylbut-2-en-1-yl diphosphate synthase (EC 1.17.4.3), 2-C-methyl-D-erythritol 2,4-cyclodiphosphate synthase (EC 4.6.1.12), 4-(cytidine 5′-diphospho)-2-C-methyl-D-erythritol kinase (EC 2.7.1.148), 2-C-methyl-D-erythritol 4-phosphate cytidylyltransferase (EC 2.7.7.60), 1-deoxy-D-xylulose-5-phosphate reductoisomerase (EC 1.1.1.267), 1-deoxy-D-xylulose-5-phosphate synthase (EC 2.2.1.7), isopentenyl-diphosphate delta-isomerase (EC 5.3.3.2), farnesyltranstransferase (EC 2.5.1.29), geranyltranstransferase (EC 2.5.1.10), dimethylallyltranstransferase (EC 2.5.1.1), diphosphomevalonate decarboxylase (EC 4.1.1.33), phosphomevalonate kinase (EC 2.7.4.2), mevalonate kinase (EC 2.7.1.36), hydroxymethylglutaryl-CoA reductase (NADPH; EC 1.1.1.34), vitamin-K-epoxide reductase (warfarin-sensitive; EC 1.1.4.1), NAD(P)H dehydrogenase (quinine; EC 1.6.5.2), phytoene synthase (EC 2.5.1.32), carotene 7,8-desaturase (EC 1.14.99.30), zeaxanthin epoxidase, neoxanthin synthase, squalene synthase (EC 2.5.1.21), trans-pentaprenyltranstransferase (EC 2.5.1.33), trans-hexaprenyl transtransferase (EC 2.5.1.30), trans-octaprenyltranstransferase (EC 2.5.1.11), squalene monooxygenase (EC 1.14.99.7), lanosterol synthase (EC 5.4.99.7), cycloartenol synthase (EC 5.4.99.8), cholestenol delta-isomerase (EC 5.3.3.5), lathosterol oxidase (EC 1.14.21.6), 7-dehydrocholesterol reductase (EC 1.3.1.21), 9-cis-epoxycarotenoid dioxygenase, short-chain dehydrogenase/reductase (SDR), aldehyde oxidase 3 (AAO3), calcidiol 1-monooxygenase (EC 1.14.13.13), or oxidoreductase (EC 1.14.13.- or EC 1.3.99.-).

In some cases, a sequence of interest encodes an enzyme involved in terpenoid biosynthesis such as aristolochene synthase (EC 4.2.3.9), trichodiene synthase (EC 4.2.3.6), (+)-delta-cadinene synthase (EC 4.2.3.13), vetispiradiene synthase (EC 4.2.3.21), di-trans,poly-cis-decaprenylcistransferase (EC 2.5.1.31), or strictosidine synthase (EC 4.3.3.2).

In some cases, a sequence of interest encodes an enzyme involved in monoterpenoid biosynthesis such as (4S)-limonene synthase (EC 4.2.3.16), (R)-limonene synthase (EC 4.2.3.20), pinene synthase (EC 4.2.3.14), (−)-endo-fenchol synthase (EC 4.2.3.10), myrcene synthase (EC 4.2.3.15), sabinene-hydrate synthase (EC 4.2.3.11), bornyl diphosphate synthase (EC 5.5.1.8), (S)-limonene 3-monooxygenase (EC 1.14.13.47), isopiperitenol dehydrogenase (EC 1.1.1.223), (−)-menthol dehydrogenase (EC 1.1.1.207), (+)-neomenthol dehydrogenase (EC 1.1.1.208), (S)-limonene 6-monooxygenase (EC 1.14.13.48), carveol dehydrogenase (EC 1.1.1.243), (S)-limonene 7-monooxygenase (EC 1.14.13.49), (R)-limonene 6-monooxygenase (EC 1.14.13.80), or (+)-trans-carveol dehydrogenase (EC 1.1.1.275).

In some embodiments, the expression level of a sequence of interest can be amplified while retaining the specificity of expression using a regulatory region described herein, such as a regulatory region set forth in any of SEQ ID NOs:1-3. The sequence of interest is operably linked to one or more recognition sites (e.g., one, two, three, four, five, six, seven, eight, nine, ten, or more recognition sites) for a transcription activator polypeptide and, optionally, a promoter. In addition, a regulatory region described herein is operably linked to a nucleotide sequence encoding a transcription activator polypeptide that binds to the recognition site operably linked to the sequence of interest, resulting in an increase in the level of transcription of the sequence of interest. The regulatory region operably linked to the nucleotide sequence encoding a transcription activator, and the sequence of interest operably linked to one or more recognition sites, can be included in the same or in different nucleic acid constructs. Populations of transgenic plants or plant cells having one or more nucleic acid constructs including a regulatory region operably linked to a nucleotide sequence encoding a transcription activator and a sequence of interest operably linked to one or more recognition sites can be produced by transformation, transfection, or genetic crossing. See, e.g., WO 97/31064.

Suitable transcription activators include, without limitation, plant transcription activators, chimeric transcription activators, and yeast transcription activators. Plant transcription activators typically are from a species that is in a different taxonomic genus from plants used in a method, are from a species that is geographically widely separated from plants used in a method, and/or are from a species where the timing or tissue specificity of naturally occurring expression differs from that occurring in plants used in a method. If desired, a transcription activator can be tested for its allergenic properties and those that are non-allergenic selected for use. Suitable transcription activators include YAP1, YAP2, SKO1, zinc finger protein M1G1, ABF1 and UME6, all of which are from yeast. Other suitable transcription activators include AtERF1, AtERF2, AtERF5, CBF1 and Athb-1, all of which are from plants. See, e.g., Fujimoto, S. Y. et al. (2000) Plant Cell 12:393-404; Stockinger, E. J. et al. (1997) Proc. Natl. Acad. Sci. USA 94:1035-40; and Aoyama, T. et al. (1995) Plant Cell 7:1773-85.

Many transcription activators have discrete DNA binding and transcription activation domains. Thus, DNA binding domain(s) and transcription activation domain(s) of a suitable transcription activator can be derived from different sources, i.e., can be a chimeric transcription activator. For example, a transcription activator can have a DNA binding domain derived from the yeast gal4 gene and a transcription activation domain derived from the VP16 gene of herpes simplex virus. In other embodiments, a transcription activator can have a DNA binding domain derived from a yeast HAP1 gene and the transcription activation domain derived from VP16. In yet other embodiments, a transcription activator can have a DNA binding domain derived from a yeast gal4 or HAP1 gene and a transcription activation domain derived from a maize C1 gene. See, e.g., Guyer et al., Genetics 149:633-639 (1998). Transcription activation domains from the maize DOF1 and rice RISBZ1 transcription activators can also be used in a chimeric transcription activator. Table 1 below sets forth other plant transcription activation domains that can be used in a chimeric transcription activator.

TABLE 1
Transcription Activation Domains

| Source | Organism | Amino Acids | Reference |
|---|---|---|---|
| C1 protein | Maize | 173-273; | Goff S A et al., Gene & Dev |
| | | 146-269; | (1991) 5: 298-309 |
| | | 214-273 | Van Eenennaam et al., Metab |
| | | | Eng. (2004) 6: 101-8 |
| ATMYB2 | Arabidopsis | 221-274 | Urao et al., Plant J. (1996) |
| | | | 10: 1145-8. |
| HALF-1 | Wheat | 203-256 | Okanami et al., Genes to |
| | | | Cells (1996) 1: 87-99. |
| ANT | Arabidopsis | 133-274; | Krizek & Sulli, Planta (2006) |
| | | 134-213 | 224: 612-21. |
| ALM2 | Arabidopsis | 1-163 | Anderson & Hanson, BMC Plant |
| | | | Biol. (2005) 21; 5(1): 2. |

In some embodiments, a chimeric transcription activator contains a non-naturally occurring DNA-binding domain. Non-naturally occurring domains that selectively bind to a specific DNA sequence can be generated using methods known in the art. See, e.g., U.S. Pat. No. 5,198,346.

Examples of transcription activators and their cognate recognition sites that can be used include those listed in Table 2 below, all of which are from Saccharomyces cerevisiae. See, e.g., Fernandes et al, Mol. Cell. Bio., 17:6982-93 (1997); Nehlin et al., Nucleic Acids Res., 20:5271-8 (1992); Lundin et al., Mol. Cell. Biol., 14:1979-85 (1994); Buchman et al., (1988) Mol. Cell. Biol., 8:210-225 (1988); and Williams et al. Proc Natl Acad Sci USA, 99:13431-62002 (2002).

TABLE 2

Transcription Activators and their Recognition
Sites

| | |
|---|---|
| Transcription activator | Recognition site |

| | |
|---|---|
| YAP1 and YAP2 | TTACTAA |
| SKO1 | CRE motif: TGACGTCA |
| zinc finger protein MIG1 | GGTAAAAATGCGGG |
| | (SEQ ID NO: 4) |
| ABF1 | 5′-TnnCGTnnnnnnTGAT-3′ |
| | (SEQ ID NO: 5) |
| UME6 | TSGGCGGCTAW |
| | (SEQ ID NO: 6) |

Transgenic Plants and Cells

Nucleic acids provided herein can be used to transform plant cells and generate transgenic plants. Thus, transgenic plants and plant cells containing the nucleic acids described herein also are provided, as are methods for making such transgenic plants and plant cells. A plant or plant cell can be transformed by having the construct integrated into its genome, i.e., can be stably transformed. Stably transformed cells typically retain the introduced nucleic acid sequence with each cell division. A plant or plant cell also can be transiently transformed such that the construct is not integrated into its genome. Transiently transformed cells typically lose some or all of the introduced nucleic acid construct with each cell division, such that the introduced nucleic acid cannot be detected in daughter cells after sufficient number of cell divisions. Both transiently transformed and stably transformed transgenic plants and plant cells can be useful in the methods described herein.

Transgenic plant cells used in the methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques.

As used herein, a transgenic plant also refers to progeny of an initial transgenic plant. Progeny include descendants of a particular plant or plant line. Progeny of an instant plant include seeds formed on F₁, F₂, F₃, F₄, F₅, F₆, and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅, and F₆ refer to subsequent generations of self- or sib-pollinated progeny of an F₁ plant. Seeds produced by a transgenic plant can be grown and then selfed (or outcrossed and selfed) to obtain plants and seeds homozygous for the nucleic acid construct.

Transgenic plant cells can be grown in suspension culture, or tissue or organ culture. Solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

Techniques for transforming a wide variety of higher plant species are known in the art. The polynucleotides and/or recombinant vectors described herein can be introduced into the genome of a plant host using any of a number of known methods, including electroporation, microinjection, and biolistic methods. Alternatively, polynucleotides or vectors can be combined with suitable T-DNA flanking regions and introduced into a conventional Agrobacterium tumefaciens host vector. Such Agrobacterium tumefaciens-mediated transformation techniques, including disarming and use of binary vectors, are well known in the art. Other gene transfer and transformation techniques include protoplast transformation through calcium or PEG, electroporation-mediated uptake of naked DNA, electroporation of plant tissues, viral vector-mediated transformation, and microprojectile bombardment (see, e.g., U.S. Pat. Nos. 5,538,880; 5,204,253; 5,591,616; and 6,329,571). If a cell or tissue culture is used as the recipient tissue for transformation, plants can be regenerated from transformed cultures using techniques known to those skilled in the art.

The polynucleotides and vectors described herein can be used to transform a number of dicotyledonous plants and plant cell systems, including apricot, avocado, bean, blackberry, blueberry, cantaloupe, cherry, cranberry, cucumber, currant, eggplant, gooseberry, grape, grapefruit, honeydew, lemon, melon, nectarine, orange, pea, peach, peanut, pepper, plum, pumpkin, raspberry, soybeans, strawberry, squash, tomato, walnut, and watermelon. Thus, the methods and compositions described herein can be utilized with dicotyledonous plants such as those belonging to the orders Ericales, Fabales, Juglandales, Laurales, Rosales, Rhamnales, Sapindales, Solanales, and Violales. The methods and compositions can be used over a broad range of plant species, including species from the dicot genera Arachis, Capsicum, Citrullus, Citrus, Cucumis, Cucurbita, Glycine, Juglans, Lycopersicon, Persea, Phaseolus, Pisum, Prunus, Ribes, Rubus, Solanum, Vaccinium, and Vitis.

A transformed cell, callus, tissue, or plant can be identified and isolated by selecting or screening the engineered plant material for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, quantitative PCR, or reverse transcriptase PCR(RT-PCR) amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known.

A regulatory region disclosed herein can be used to express any of a number of heterologous nucleic acids of interest in a plant. For example, a regulatory region disclosed herein can be used to express a polypeptide or an interfering RNA. Suitable polypeptides include, without limitation, polypeptides encoded by sequences of interest described above such as polypeptides involved in flavonoid, stilbene, coumarin, phytosterol, terpenoid, and/or monoterpenoid biosynthesis. Additional examples of suitable polypeptides include screenable and selectable markers such as green fluorescent protein, yellow fluorescent protein, luciferase, β-glucuronidase, or neomycin phosphotransferase II. Suitable polypeptides also include polypeptides that affect fruit size, shape, development, and maturity. In some embodiments, a heterologous nucleic acid encodes a stilbene synthase polypeptide (see, e.g., U.S. Pat. Nos. 5,500,367; 5,728,570; and 6,063,988), or an enzyme involved in saccharide formation, e.g., levansucrase, dextransucrase, invertase, or sucrose phosphate synthase. In some embodiments, a heterologous polynucleotide encodes a non-plant polypeptide of pharmaceutical or industrial interest. In some embodiments, a heterologous nucleic acid encodes a polypeptide involved in pest defense, such as a Bacillus thuringiensis (Bt) insecticidal polypeptide. In some cases, a regulatory region disclosed herein can be used to express a cyclin polypeptide, such as a cyclin polypeptide encoded by a CYC1 gene, in the fruit of a plant. In some cases, a regulatory region disclosed herein can be used to express an interfering RNA that inhibits transcription of an expansin gene, such as LeExp1, in the fruit of a plant. Expression of such a polypeptide or interfering RNA can affect the phenotype of a plant, e.g., a transgenic plant, when expressed in the plant, e.g., at the appropriate time(s), in the appropriate tissue(s), or at the appropriate expression levels. Thus, transgenic plants, plant organs, plant tissues, or plant cells can have an altered phenotype as compared to corresponding control plants, plant organs, plant tissues, or plant cells, respectively, that either lack the transgene or do not express the transgene. A corresponding control plant can be a corresponding wild-type plant, a corresponding plant that is not transgenic but otherwise is of the same genetic background as the transgenic plant of interest, or a corresponding plant of the same genetic background in which expression of the transgene is suppressed, inhibited, or not induced, e.g., where expression is under the control of an inducible promoter. A plant can be said “not to express” a transgene when the plant exhibits less than 10%, e.g., less than 9%, 8%, 7%, 6%, 5%, 4%, 3%, 2%, 1%, 0.5%, 0.1%, 0.01%, or 0.001%, of the amount of the polypeptide, mRNA encoding the polypeptide, or transcript of the transgene exhibited by the plant of interest. Expression can be evaluated using methods including, for example, quantitative PCR, RT-PCR, Northern blots, S1 RNase protection, primer extensions, Western blots, protein gel electrophoresis, immunoprecipitation, enzyme-linked immunoassays, microarray technology, and mass spectrometry. It should be noted that if a transgene is expressed under the control of a tissue-preferential or broadly expressing promoter, expression can be evaluated in a selected tissue or in the entire plant. Similarly, if a transgene is expressed at a particular time, e.g., at a particular time during development or upon induction, expression can be evaluated selectively during a desired time period.

Use of a regulatory region provided herein to direct expression of a cyclin gene, such as a CYC1 gene, in the fruit of a plant can increase sink strength and fruit yield compared to a corresponding control plant. In some embodiments, use of a regulatory region described herein to express a gene in fruit that is involved in the regulation of cell expansion (e.g., through effects on brassinosteroid response pathways), such as a Brassinazole Resistant 1 (BZR1) gene, may allow production of crops with increased fruit size compared to corresponding control crops. In some embodiments, use of a regulatory region provided herein to express a sucrose phosphate synthase polypeptide in the fruit of a plant can increase the sweetness of the fruit and the Brix value of juice or wine produced from the fruit. In some embodiments, use of a regulatory region described herein to inhibit expression of a sucrose phosphate synthase gene can decrease the sweetness and caloric content of the fruit compared to the fruit of a corresponding control plant. In some embodiments, use of a regulatory region provided herein to express resveratrol synthase in the peel of a fruit can increase the level of resveratrol compared to that in the peel of a corresponding control fruit. In some embodiments, use of the materials and methods described herein to inhibit expression in fruit of an expansin gene, such as LeExp1, or a gene involved in regulation of ethylene-inducible genes and pathways, such as a gene encoding an Ethylene-Response DNA-Binding Factor (EDF) transcription factor polypeptide, can delay fruit ripening and extend the shelf life of the fruit compared to the shelf life of corresponding control fruit.

Seeds of transgenic plants describe herein can be conditioned and bagged in packaging material by means known in the art to form an article of manufacture. Packaging material such as paper and cloth are well known in the art. Such a bag of seed preferably has a package label accompanying the bag, e.g., a tag or label secured to the packaging material, a label printed on the packaging material, or a label inserted within the bag.

The invention will be further described in the following examples, which do not limit the scope of the invention described in the claims.

### EXAMPLES

### Example 1

### Strategy for Identification of Tissue Preferential Regulatory Regions

A strategy was conceived to identify tissue preferential regulatory regions. The strategy involves using a regulatory region that directs transcription preferentially in a particular tissue of an organism of interest to identify additional regulatory regions that direct transcription preferentially in the same tissue of the organism of interest. The regulatory region that directs transcription preferentially in a particular tissue of an organism of interest is operably linked to a reporter gene and introduced into another organism, such as a model organism. The model organism is then studied to determine which tissue(s) express the reporter gene operably linked to the regulatory region. The tissue expression profile of the regulatory region in the model organism is compared to the tissue expression profiles of other regulatory regions in the model organism. Regulatory regions are selected that produce expression profiles in the model organism that are similar to the expression profile produced in the model organism by the regulatory region that directs transcription in a tissue preferential manner in an organism of interest. Each of the selected regulatory regions is operably linked to a reporter gene, and the expression profile of the reporter gene directed by each regulatory region is investigated in the organism of interest to confirm that the selected regulatory region directs transcription preferentially in the same tissue of the organism of interest as the tissue preferential regulatory region used to select the additional regulatory regions.

### Example 2

### Identifying Fruit Preferential Regulatory Regions

The strategy described in Example 1 above was implemented to identify tomato fruit preferential regulatory regions. A regulatory region, PG800, containing 806 bp of the 5′ flanking sequence of a polygalacturonase gene, directs transcription preferentially in tomato fruit (Montgomery et al., Plant Cell, 5:1049-1062 (1993)). The PG800 regulatory region was cloned using the polymerase chain reaction (PCR), and the PCR clone was confirmed by DNA sequencing.

The PG800 regulatory region was operably linked to a luciferase reporter gene in a nucleic acid construct that was used to generate transgenic Arabidopsis plants essentially as described in Bechtold et al., C.R. Acad. Sci. Paris, 316:1194-1199 (1993). Four transgenic Arabidopsis lines were generated from independent transformation events. The expression pattern of the luciferase reporter gene operably linked to PG800 was analyzed in the transgenic Arabidopsis plants using bioluminescence imaging (NightOWL™ LB 981 system, Berthold Technologies, Oak Ridge, Tenn.). Expression of luciferase directed by the PG800 regulatory region was observed predominantly in the flowers of the transgenic Arabidopsis plants. The observed expression of luciferase in the flower included expression in the petal and sepal. Luciferase expression was also observed in green, but not dry, seed. No expression of luciferase was observed in seedlings, siliques, roots, rosettes, or upper sterns. A very low intensity of luciferase expression could be observed in the lower stem.

Arabidopsis regulatory regions were screened to determine their tissue expression profiles in Arabidopsis plants. Each regulatory region tested was cloned into a vector including a two-component expression system such that it was operably linked to a synthetic HAP1-VP16 gene. The HAP1-VP16 gene included a nucleic acid encoding a DNA binding domain of a yeast HAP1 zinc finger transcription factor polypeptide fused to a nucleic acid encoding a transcriptional activation domain of a herpes simplex virus VP16 polypeptide. The vector construct also included a HAP1 upstream activation sequence operably linked to a GFP gene such that GFP would be expressed in response to expression of the HAP1-VP16 polypeptide. The GFP gene was optimized for expression in plants. See, e.g., U.S. Patent Publication No. 20050132432. Each vector construct containing a regulatory region was used to transform Arabidopsis plants essentially as described in Bechtold et al., C.R. Acad. Sci. Paris, 316:1194-1199 (1993).

Three Arabidopsis regulatory regions were selected that had expression profiles similar to the expression profile of PG800 in Arabidopsis. The regulatory region PT0623 (genomic locus At3g22840; SEQ ID NO:1) was observed to produce a high intensity of GFP expression in flowers and roots of transgenic Arabidopsis plants. The regulatory region YP0396 (genomic locus At5g52390; SEQ ID NO:2) was observed to produce a high intensity of GFP expression in flowers and leaves of transgenic Arabidopsis plants. The regulatory region YP0377 (genomic locus At1g07135; SEQ ID NO:3) was observed to produce an intermediate intensity of GFP expression in flowers and roots of transgenic Arabidopsis plants.

Each vector construct described above containing PT0623 (PT0623::GFP), YP0396 (YP0396::GFP), or YP0377 (YP0377::GFP) was used independently to transform cotyledon explants from cherry tomato seedlings, essentially as described elsewhere (Park et al., J Plant Physiol., 160:1253-1257 (2003)). Cotyledon explants from cherry tomato seedlings were also transformed with the construct containing the PG800 regulatory region operably linked to a luciferase reporter gene.

Transgenic tomatoes grown from cotyledon explants transformed with the construct containing the Arabidopsis regulatory region PT0623 or YP0396 were analyzed for GFP expression. A high intensity of GFP expression was observed in transgenic cherry tomato fruit grown from cotyledon explants transformed with the PT0623::GFP or the YP0396::GFP construct.

Expression of the PT0623::GFP construct was examined at different stages of fruit ripening in transgenic cherry tomatoes. Fruit preferential expression of GFP was observed using fluorescence scanning (Typhoon™ imaging system, GE Healthcare Bio-Sciences Corp., Piscataway, N.J.). The pattern and intensity of GFP expression observed in the transgenic fruit was similar in two out of three transformation events. Although the intensity of GFP was weaker in the third event, the pattern of expression was nevertheless similar to that of the other two events. GFP expression was also analyzed in fruit from PT0623::GFP transgenic plants using confocal microscopy. GFP expression was observed in the flesh tissues of the fruit, but not in the seed of the transgenic cherry tomatoes. GFP expression also was observed in the flesh tissue remaining attached to the inside layer of the peel following removal of the peel from the tomato, but little or no GFP expression was observed in the peel itself.

Cherry tomatoes transformed with the YP0396::GFP construct also were examined for GFP expression using fluorescence scanning and confocal microscopy. The regulatory region YP0396 was observed to produce a pattern of GFP expression similar to that produced by the PT0623 regulatory region in transgenic cherry tomatoes. However, the intensity of GFP expression produced by the YP0396 regulatory region was slightly lower than that produced by the PT0623 regulatory region. In addition, confocal microscopic analysis of cross sections of the peel indicated that the YP0396 regulatory region produced a very high intensity of GFP expression in cell layers of the peel of transgenic cherry tomatoes from two out of three events tested.

T₁ transgenic tomato plants gown from seed produced by regenerated T₀ plants transformed with the YP0377::GFP construct also were analyzed for GFP expression using fluorescence scanning. Transgenic plants from two transformation events were examined. Fruit preferential expression of GFP was observed in the transgenic tomato plants using fluorescence scanning. An intermediate intensity of GFP expression was observed in the fruit of green cherry tomatoes transformed with the YP0377::GFP construct. The observed intensity of the GFP expression was much weaker than that produced by the PT0623::GFP construct, and stronger than the GFP intensity produced by the YP0396::GFP construct. In addition, a weak to intermediate intensity of GFP expression was produced by the YP0377 regulatory region in the stems of plants from one of the two events analyzed.

Transgenic cherry tomato plants from independent transformations with the control construct containing the PG800 regulatory region operably linked to a luciferase gene were analyzed for luciferase expression. Fruit specific expression of luciferase was detected in cherry tomato fruit from five out of eight independent transformation events with the control construct. No luciferase expression was observed in untransformed control plants.

### Example 3

### Expression of GFP in Tomato Plants Using the PT0623 and YP0396 Regulatory Regions

The vector constructs described above containing PT0623::GFP or YP0396::GFP were digested overnight with PstI or BamHI restriction enzyme (Invitrogen, Carlsbad, Calif.), respectively, at 37° C. to remove the HAP1-VP16 two component activation system. The digested fragments in each reaction were separated by 1% agarose gel electrophoresis in 1×TBE (Tris Borate EDTA) buffer. Each of the bands corresponding to the digested PT0623::GFP and YP0396::GFP vector backbones was removed from the agarose gel, eluted using the Qiagen gel purification kit (Qiagen, Valencia, Calif.), self ligated using T4 ligase (Invitrogen), and transformed into TOP10 competent E. coli cells (Invitrogen) by the heat shock method. Colonies were selected and plasmid sequences were confirmed by DNA sequencing. The plasmids were designated PT0623::GFP-DF or YP0396:GFP-DF.

Each of the PT0623::GFP-DF and YP0396:GFP-DF constructs was used to transform cotyledon explants from cherry tomato seedlings as described above. To transgenic tomato plants regenerated from the explants were analyzed for GFP expression using fluorescence scanning as described above. Cherry tomato fruit from the transgenic plants also was analyzed for GFP expression. The results are presented in Table 3 below.

TABLE 3
GFP expression produced by the PT0623 and YP0396
regulatory regions in transgenic tomato plants

| | GFP expression in leaf, stem, | GFP expression in green, |
|---|---|---|
| Transgenic Plant | flower, and/or fruit | yellow, orange, or red fruit |
| PT0623::GFP-DF, event 5 | little to no expression detected | little to no expression detected |
| PT0623::GFP-DF, event 9 | little to no expression detected | low to moderate expression in |
| | | peel |
| PT0623::GFP-DF, event 11 | low to moderate expression in | low to moderate expression |
| | peel | |
| YP0396::GFP-DF, event 3 | little to no expression detected | low to moderate expression |
| | | moderate expression detected as |
| | | fruit ripened |
| YP0396::GFP-DF, event 7 | little to no expression detected | low to moderate expression |
| | | moderate expression detected as |
| | | fruit ripened |
| YP0396::GFP-DF, event 10 | little to no expression detected | low to moderate expression |
| | | moderate expression detected as |
| | | fruit ripened |

Based on the GFP expression profiles of events 11 and 8 containing PT0623::GFP-DF and YP0396::GFP-DF, respectively, the PT0623 and YP0396 regulatory regions produce higher expression levels in fruit than in other tissues, and the expression levels in fruit tend to become stronger as the fruit ripens. Use of the PT0623 or YP0396 regulatory region to drive expression of GFP in a two component expression system, as described in Example 2 above, produced a higher intensity of GFP expression in tomato fruit than use of either regulatory region to drive expression of GFP directly. In all cases, GFP expression produced by the PT0623 and YP0396 regulatory regions was observed to be fruit preferential.

### Example 4

### Deletion Analysis of the YP0396 Regulatory Region

The YP0396 regulatory region was analyzed by making 5′-deletion mutants ranging from 225-766 base pairs in length. Each deletion mutant was cloned into a vector and used to generate Arabidopsis plants as set forth in Example 2. As indicated in Table 4, the expression profile of YP0396 (SEQ ID NO:2) was maintained with YP0396 fragments containing nucleotides 500-1000 of SEQ ID NO: 2 (YP0396-d (˜500) (also called YP2225), nucleotides 350-1000 of SEQ ID NO: 2 (YP0396-d(˜650), and nucleotides 234-1000 of SEQ ID NO:2 (YP0396-d(˜766)). No expression was observed with YP0396 fragments 225-433 nucleotides in length,

TABLE 4
GFP expression produced by YP0396 regulatory
regions in transgenic Arabidopsis plants

| Transgenic plant | Length | Expression |
|---|---|---|
| YP0396 | 1000 | Anther, style, ovule, seed coat |
| YP0396-d(˜766) | 766 | Anther, style, ovule, seed coat |
| YP0396-d(˜650) | 650 | Anther, style, ovule, seed coat |
| YP0396-d(˜500) (also | 500 | Anther, style, ovule, seed coat |
| called YP2225) | | |
| YP0396-d(˜433) | 433 | No expression |
| YP0396-d(˜350) (also | 350 | No expression |
| called YP2224) | | |
| YP0396-d(˜225)(also | 225 | No expression |
| called YP2221) | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid having 90% or greater sequence identity to a polynucleotide sequence selected from the group consisting of a) nucleotides 500 to 1000 of SEQ ID NO:2, b) nucleotides 350 to 1000 of SEQ ID NO:2, and c) nucleotides 234 to 1000 of SEQ ID NO:2, wherein said nucleic acid is 500 to 800 nucleotides in length and has the ability to direct transcription in fruit tissues of a plant.

2. The isolated nucleic acid of claim 1, wherein said sequence identity is 95% or greater.

3. The isolated nucleic acid of claim 1, wherein said sequence identity is 99% or greater.

4. A nucleic acid construct comprising a regulatory region operably linked to a heterologous polynucleotide, wherein said regulatory region is 500 to 800 nucleotides in length and has 90% or greater sequence identity to the polynucleotide sequence selected from the group consisting of a) nucleotides 500 to 1000 of SEQ ID NO:2, b) nucleotides 350 to 1000 of SEQ ID NO:2, and c) nucleotides 234 to 1000 of SEQ ID NO:2.

5. The nucleic acid construct of claim 4, wherein said sequence identity is 95% or greater.

6. The nucleic acid construct of claim 4, wherein said sequence identity is 99% or greater.

7. The nucleic acid construct of claim 4, wherein said heterologous polynucleotide comprises a nucleotide sequence encoding a polypeptide.

8. The nucleic acid construct of claim 7, wherein said polypeptide is a resveratrol synthase polypeptide.

9. The nucleic acid construct of claim 7, wherein said polypeptide is an enzyme involved in flavonoid biosynthesis.

10. The nucleic acid construct of claim 7, wherein said polypeptide is an enzyme involved in phytosterol biosynthesis.

11. The nucleic acid construct of claim 7, wherein said polypeptide is an enzyme involved in monoterpenoid biosynthesis.

12. The nucleic acid construct of claim 4, wherein said heterologous polynucleotide is in an antisense orientation relative to said regulatory region.

13. The nucleic acid construct of claim 4, wherein said heterologous polynucleotide is transcribed into an interfering RNA.

14. A transgenic plant or plant cell comprising a nucleic acid construct, said nucleic acid construct comprising a regulatory region operably linked to a heterologous polynucleotide, wherein said regulatory region is 500 to 800 nucleotides in length and has 90% or greater sequence identity to the polynucleotide sequence selected from the group consisting of a) nucleotides 500 to 1000 of SEQ ID NO:2, b) nucleotides 350 to 1000 of SEQ ID NO:2, and c) nucleotides 234 to 1000 of SEQ ID NO:2.

15. The transgenic plant or plant cell of claim 14, wherein said plant or plant cell is from a genus selected from Capsicum, Fragaria, Lycopersicon, Solanum, Vaccinium, or Vitis.

16. A transgenic plant or plant cell comprising (a) a first nucleic acid comprising a regulatory region operably linked to a heterologous polynucleotide encoding a transcription activator polypeptide, wherein said regulatory region is 500 to 800 nucleotides in length and has 90% or greater sequence identity to the polynucleotide sequence selected from the group consisting of i) nucleotides 500 to 1000 of SEQ ID NO:2, ii) nucleotides 350 to 1000 of SEQ ID NO:2, and iii) nucleotides 234 to 1000 of SEQ ID NO:2, and (b) a second nucleic acid comprising a sequence of interest operably linked to a recognition site for said transcription activator polypeptide.

17. The transgenic plant or plant cell of claim 16, wherein said sequence of interest encodes a polypeptide.

18. The transgenic plant or plant cell of claim 17, wherein said polypeptide is a resveratrol synthase polypeptide.

19. The transgenic plant or plant cell of claim 16, wherein said first and second nucleic acid are present on the same nucleic acid construct.

20. The transgenic plant or plant cell of claim 16, wherein said plant or plant cell is from a genus selected from Capsicum, Fragaria, Lycopersicon, Solanum, Vaccinium, or Vitis.

21. A method of producing a transgenic plant, said method comprising (a) introducing into a plant cell the nucleic acid construct of claim 4; and (b) growing a plant from said plant cell.

22. A method of identifying whether or not a regulatory region has a desired expression profile in an organism of interest, said method comprising:
(a) transforming a model organism with an isolated nucleic acid comprising a first regulatory region operably linked to a reporter gene, said first regulatory region having said desired expression profile in said organism of interest;
(b) selecting a second regulatory region having an expression profile in said model organism that is similar to the expression profile of said first regulatory region in said model organism; and
(c) determining the expression profile of said second regulatory region in said organism of interest, thereby identifying whether or not said second regulatory region has said desired expression profile in said organism of interest.

23. A method of making a transgenic plant comprising:
a) transforming plant cells with a nucleic acid construct comprising a regulatory region having 90% or greater sequence identity to the polynucleotide sequence set forth in SEQ ID NO:1, SEQ ID NO:2, or SEQ ID NO:3 operably linked to a heterologous polynucleotide, wherein said regulatory region preferentially directs transcription of said heterologous polynucleotide in fruit of said plant; and
b) identifying a plant transformed with said nucleic acid construct, thereby producing said transgenic plant, wherein said regulatory region preferentially directs transcription of said heterologous polynucleotide in fruit of said transgenic plant.

24. The method of claim 23, wherein step b) comprises identifying a plurality of plants transformed with said nucleic acid construct.

25. A method of expressing a polynucleotide in fruit tissue, comprising:
a) growing a plant under conditions in which fruit are formed, said plant comprising a nucleic acid construct comprising a regulatory region having 90% or greater sequence identity to the polynucleotide sequence set forth in SEQ ID NO:1, SEQ ID NO:2, or SEQ ID NO:3 operably linked to a heterologous polynucleotide, wherein said regulatory region preferentially directs transcription of said heterologous polynucleotide in fruit of said plant.

26. The method of claim 25, wherein said plant is from the Solanaceae family.

27. The method of claim 25, wherein said plant is tomato.

28. The method of claim 25, wherein said regulatory region preferentially directs transcription of said heterologous polynucleotide in peel tissue of said fruit.

29. The method of claim 25, wherein said regulatory region preferentially directs transcription of said heterologous polynucleotide in fleshy tissue of said fruit.

30. The method of claim 23, wherein said transcription occurs during mature green stages, breaker stage, or ripening stages of said fruit.

31. The method of claim 27, wherein said regulatory region preferentially directs transcription of said heterologous polynucleotide in peel tissue of said fruit.

32. The method of claim 27, wherein said regulatory region preferentially directs transcription of said heterologous polynucleotide in fleshy tissue of said fruit.

33. The method of claim 27, wherein said transcription occurs during mature green stages, breaker stage, or ripening stages of said fruit.
